(19) United States
(12) Patent Application Publication
(10) Pub. No.: US 2025/0041150 A1
(43) Pub. Date: Feb. 6, 2025

(54) HAND MASSAGER WITH BLOWING FUNCTION

(71) Applicant: Xiamen Vanfly Health Science & Technology Co.,Ltd., Xiamen (CN)

(72) Inventors: Qixian LIN, Xiamen (CN); Ying CHEN, Xiamen (CN)

(73) Assignee: Xiamen Vanfly Health Science & Technology Co.,Ltd., Xiamen (CN)

(21) Appl. No.: 18/429,508

(22) Filed: Feb. 1, 2024

(30) Foreign Application Priority Data
Aug. 4, 2023 (CN) 202310978168.9

(51) Int. Cl. A61H 15/00

(52) U.S. Cl. CPC A61H 15/0085; A61H 2201/0103; A61H 2201/0153; A61H 2201/0214; A61H 2201/025; A61H 2201/1207; A61H 2201/1635; A61H 2201/50; A61H 2205/065

(57) ABSTRACT

A hand massager with a blowing function, relating to the technical field of massagers. The hand massager with the blowing function includes a body, a massage mechanism and a blowing mechanism, wherein the body includes an upper housing, a lower housing and a support disposed between the two housings, both ends of the support are provided with an inlet for allowing a hand to enter and an air vent located at a finger end, and a massage area is formed between the inlet and the air vent; the massage mechanism is disposed in the massage area and configured to massage a hand; and the blowing mechanism is mounted close to a position of the air vent, and includes a blower fan, a front cover and an air duct cover adapted and connected to the front cover.

### CROSS REFERENCE TO RELATED APPLICATIONS

This application is based upon and claims foreign priority to Chinese Patent Application No. 202310978168.9, filed on Aug. 4, 2023, the entire contents of which are incorporated herein by reference.

### TECHNICAL FIELD

The present disclosure relates to the technical field of massagers, in particular to a hand massager with a blowing function.

### BACKGROUND

Since a large number of acupuncture points are concentrated in human hands, Chinese medicine considers that blood circulation can be effectively promoted by massaging and stimulating the corresponding acupuncture points, which is beneficial to physical and mental health. Therefore, with the improvement of material conditions, a demand for a hand massager also increases, and when the existing massager is of a sealed structure except for an opening through which a hand enters and exits, particularly when the existing massager is used in summer, the environment of the hand is stuffy, a hand of a user is liable to sweat, the inside of the massager is in a constant stuffy and humid state, and thus, the user is liable to feel discomfort. In order to improve this problem, a hand massager using a fan for blowing to dissipate heat has been introduced in the market, the fan directly blows to a palm of a hand, its blowing area is small, an air flow is not substantially felt except for the palm of the hand, and in addition, a cloth cover is disposed between the fan and the palm of the hand, and thus, the heat dissipation effect is not obvious.

### SUMMARY

The present disclosure discloses a hand massager with a blowing function, and aims to improve the problem of uncomfortable massage caused by stuffiness inside the existing massager.

The present disclosure employs the following solutions:

a hand massager with a blowing function includes a body, a massage mechanism and a blowing mechanism, wherein the body includes an upper housing, a lower housing and a support disposed between the two housings, both ends of the support are provided with an inlet for allowing a hand to enter and an air vent located at a finger end, and a massage area is formed between the inlet and the air vent; the massage mechanism is disposed in the massage area and configured to massage a hand; and the blowing mechanism is mounted close to a position of the air vent, and includes a blower fan, a front cover and an air duct cover adapted and connected to the front cover, an acceleration flow passage is formed between the front cover and the air duct cover, an outlet of the blower fan communicates with the acceleration flow passage, an air outlet of the acceleration flow passage is configured into a slit shape, and an air flow blown by the blower fan flows through the acceleration flow passage and is accelerated to flow out of the air outlet, so that a pressure difference is formed inside and outside of the air vent, thereby introducing air from a periphery of the air vent into the massage area to form air flow circulation.

As a further improvement, a middle part of the front cover and a middle part of the air duct cover are provided with an air inlet communicating with the air vent in a penetrating manner, the acceleration flow passage is annularly disposed at a front end of the air vent, and the air outlet is disposed toward an inner side of the air vent.

As a further improvement, the air duct cover is adapted and connected to the support, and one end of the air duct cover extends into the air vent, and the other end of the air duct cover protrudes around the air inlet to form a first air outlet portion, and the first air outlet portion is at least partially arranged to be bent from a side close to the air inlet to a side away from the air inlet.

As a further improvement, the front cover is provided with a second air outlet portion protruding around a periphery of the air inlet, the second air outlet portion is configured to be adapted to the first air outlet portion to form the air outlet, and a plurality of fins configured to abut against a curved portion of the first air outlet portion are arranged at an inner side of the second air outlet portion at intervals such that the fins divide the air outlet into a plurality of gas passages.

As a further improvement, the air outlet has a width of 1-2 mm.

As a further improvement, the massage mechanism includes an upper air bag, a lower air bag and a massage head which are disposed inside the support, the upper air bag and the lower air bag are connected with an air pump via an air tube, and the massage head is adapted and connected to a motor via a connecting piece.

As a further improvement, the blower fan, the air pump and the motor are electrically connected to a control, and the control is operatively triggered to drive the blower fan to blow air, the upper air bag and the lower air bag to inflate and deflate, and the massage head to rotate or swing up and down.

As a further improvement, the massage head is arranged on the same side as the lower air bag, the massage head is provided with a plurality of massage parts in an array in a circumferential direction, and the lower air bag is provided with a plurality of convex parts facing an inner side of the massage area.

By adopting the above technical solutions, the present disclosure can achieve the following technical effects:

in the present disclosure, an air flow is generated inside the acceleration flow passage by the blower fan, the air flow is accelerated via the slit-like air outlet of the acceleration flow passage, if a flow rate is high, a pressure will be low according to the Bernoulli's principle, a low pressure is formed inside the air vent by means of a high-speed air flow, so that air at a peripheral side of the air vent is introduced into the massage area. Air is introduced into the massage area from the front end of the air vent, and flows out through the hand inlet, thus forming continuous air flow circulation. Thus, a cool air flow is generated in the massage area, and the problems such as stuffiness and burning hot during massage are effectively relieved by blowing air to cool the temperature. Especially when a weather is hot, for people with more sweaty hands, the air flow carries away heat from the hands, making the hands feel cool and comfortable. In addition, the air flow circulation enables ventilation in the massage area, which can take away heat generated by the operation of the massager and avoid a stuffy feeling caused by the accumulation of hot air. A user can enjoy the massage while feeling the cool air flow, making the entire use process more comfortable.

### BRIEF DESCRIPTION OF THE DRAWINGS

In order to illustrate the technical solutions of the embodiments of the present disclosure more clearly, the drawings required to be used in the embodiments will be briefly described below, and it should be understood that the following drawings illustrate only certain embodiments of the present disclosure and therefore should not be regarded as limiting the scope, and that other related drawings can also be obtained from these drawings without inventive steps for those of ordinary skill in the art.

FIGS. 1 and 2 are structural schematic diagrams of a hand massager according to an embodiment of the present disclosure at different viewing angles;

FIG. 3 is a cross-sectional view of the hand massager according to the embodiment of the present disclosure;

FIGS. 4 and 5 are structural schematic diagrams of the hand massager after an upper housing and a lower housing are hidden according to the embodiment of the present disclosure at different viewing angles;

FIG. 6 is a structural schematic diagram of a blowing mechanism according to an embodiment of the present disclosure;

FIG. 7 is a cross-sectional view of FIG. 6 along one of its sections;

FIG. 8 is an exploded view of FIG. 6;

FIG. 9 is a structure schematic diagram of FIG. 6 at another viewing angle;

FIGS. 10 and 11 are structural schematic diagrams of a front cover according to an embodiment of the present disclosure at different viewing angles;

FIGS. 12 and 13 are structural schematic diagrams of an air duct cover according to an embodiment of the present disclosure at different viewing angles.

### REFERENCE NUMERALS

11—upper housing; 12—lower housing; 13—support; 131—inlet; 132—air vent; 14—decorative cover;
21—upper air bag; 22—lower air bag; 221—convex part; 23—massage head; 231—massage part; 24—air tube; 25—air pump; 26—motor; 27—wrist air bag;
31—blower fan; 32—front cover; 321—second air outlet portion; 322—fin; 33—air duct cover; 331—first air outlet portion; 34—air outlet; and 35—air inlet.
41—control; 42—button; and 43—battery.

### DETAILED DESCRIPTION OF THE EMBODIMENTS

In order to make the objects, technical solutions and advantages of the embodiments of the present disclosure more clear, the technical solutions of the embodiments of the present disclosure will be clearly and completely described below in conjunction with the accompanying drawings of the embodiments of the present disclosure, and obviously, the described embodiments are some, but not all, embodiments of the present disclosure. Based on the embodiments of the present disclosure, all other embodiments obtained by those of ordinary skill in the art without inventive steps belong to the scope of protection of the present disclosure. Thus, the following detailed description of the embodiments of the present disclosure provided in the drawings is not intended to limit the scope of the claimed disclosure, but is merely representative of the selected embodiments of the present disclosure. Based on the embodiments of the present disclosure, all other embodiments obtained by those of ordinary skill in the art without inventive steps belong to the scope of protection of the present disclosure.

### EMBODIMENTS

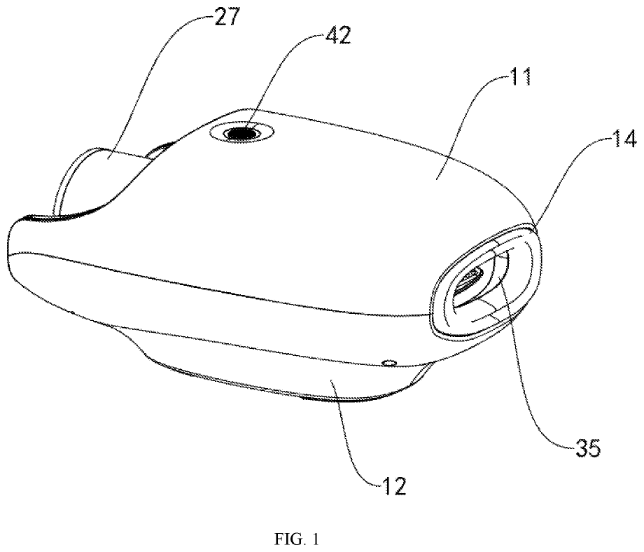

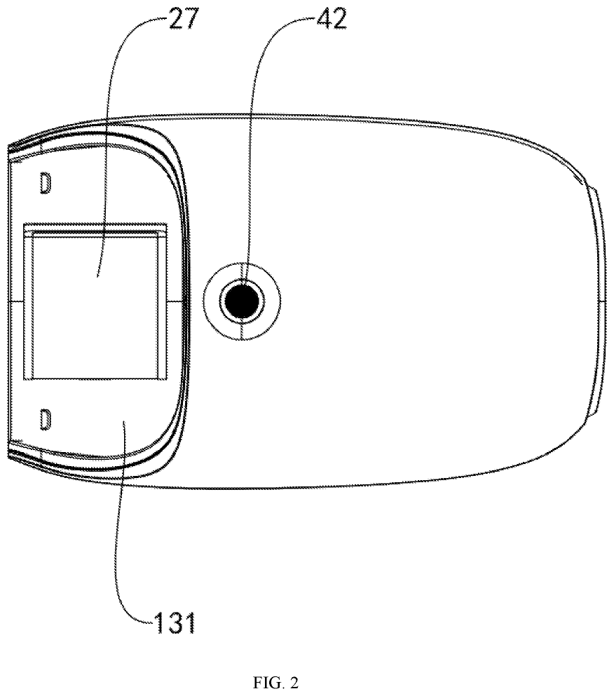

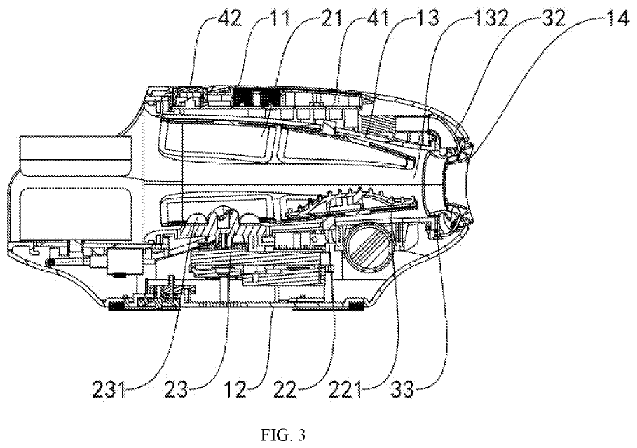

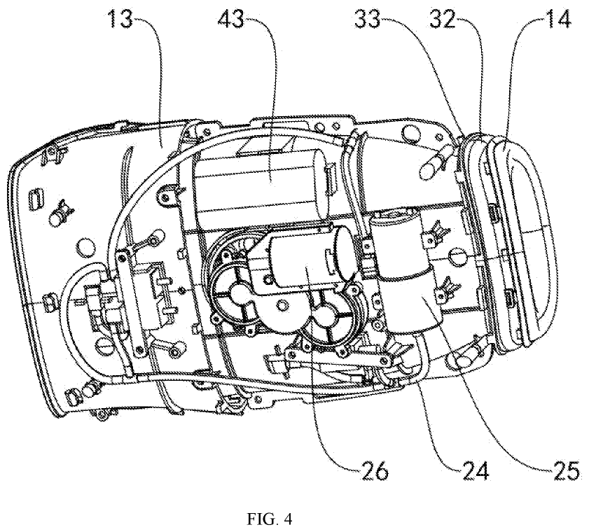

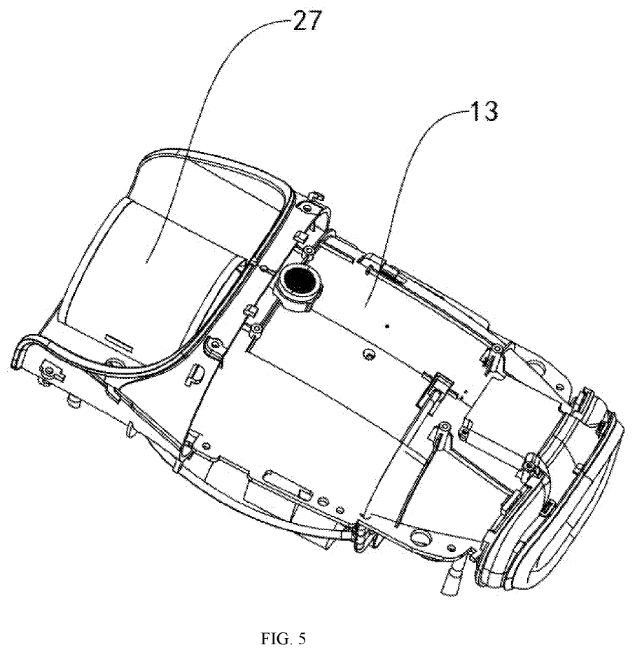

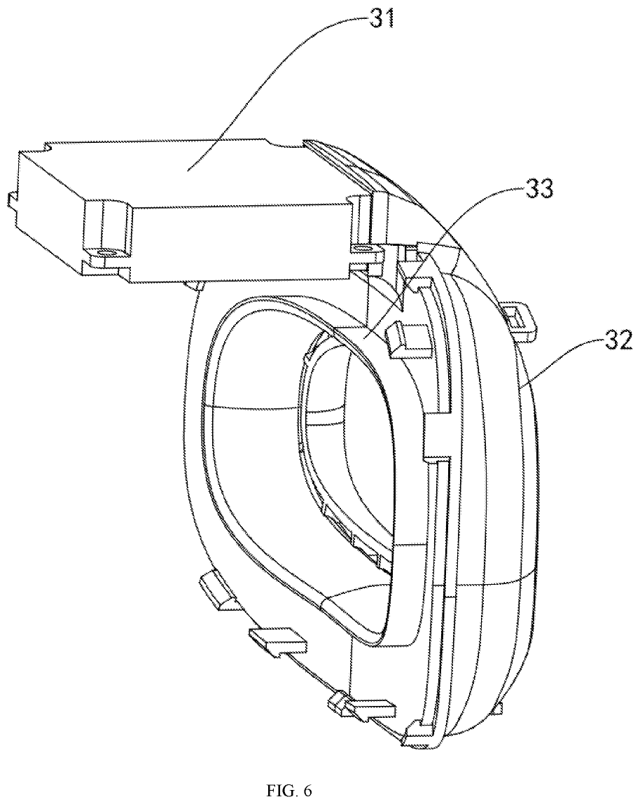

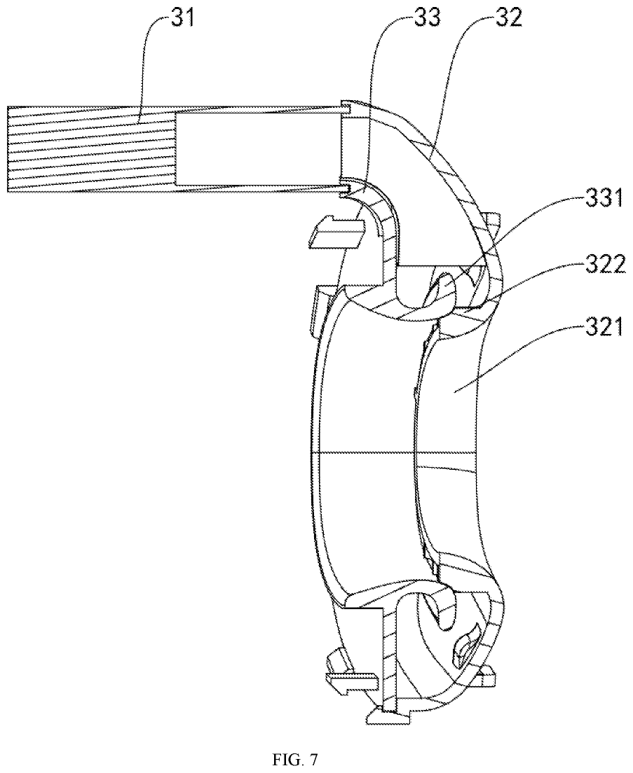

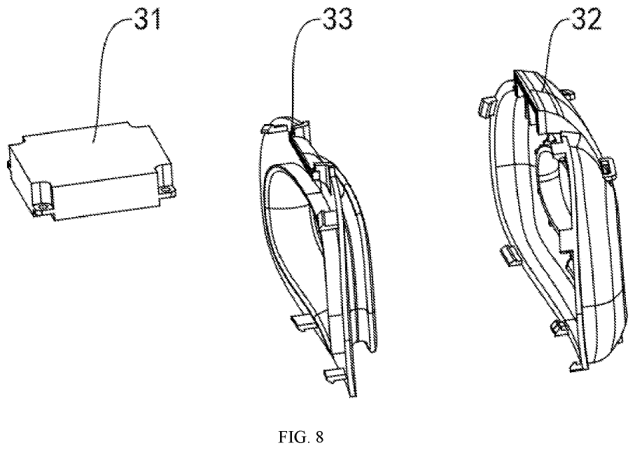

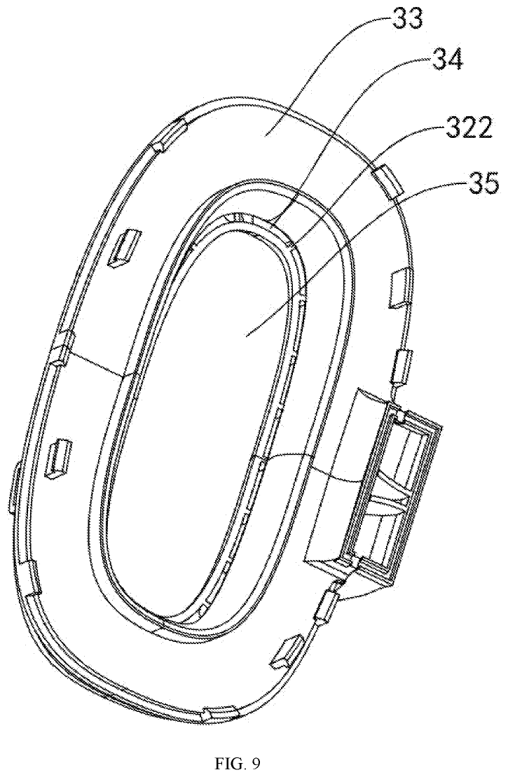

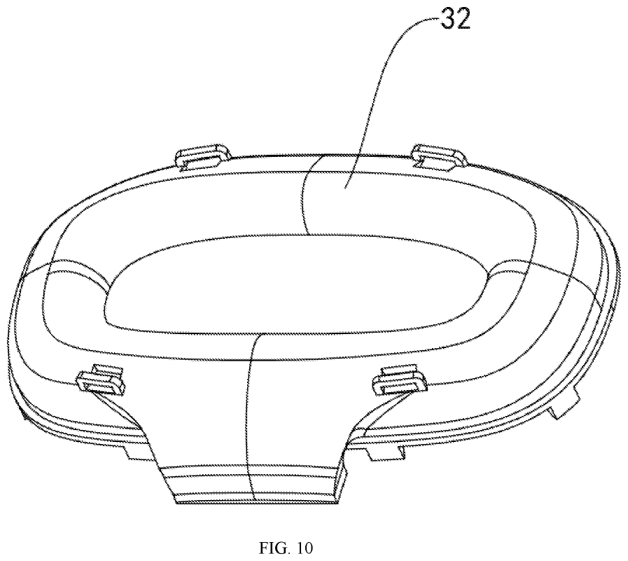

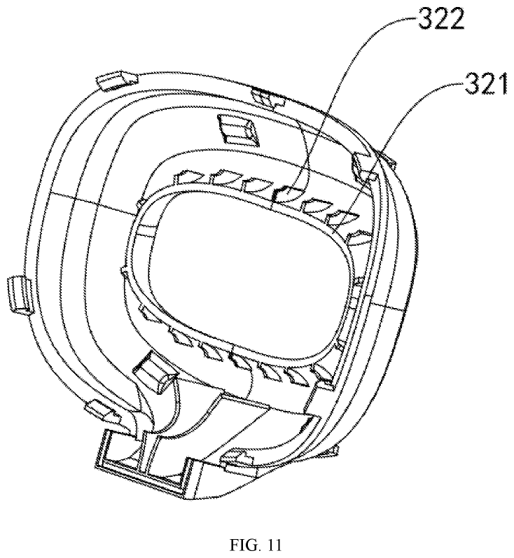

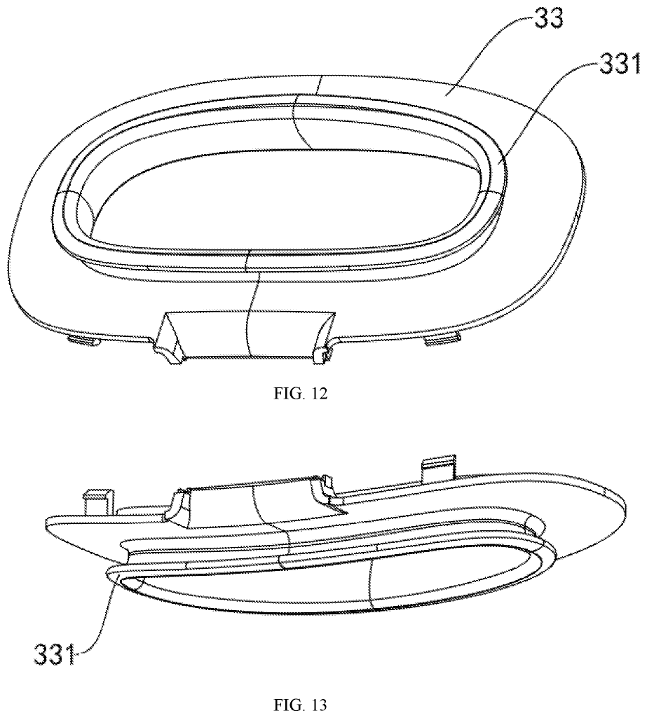

In connection with FIGS. 1 to 13, this embodiment provides a hand massager with a blowing function, including a body, a massage mechanism and a blowing mechanism, wherein the body includes an upper housing 11, a lower housing 12 and a support 13 disposed between the two housings, both ends of the support 13 is provided with an inlet 131 for allowing a hand to enter and an air vent 132 located at finger end, and a massage area is formed between the inlet 131 and the air vent 132; the massage mechanism is disposed in the massage area and configured to massage a hand; and the blowing mechanism is mounted close to a position of the air vent 132, and includes a blower fan 31, a front cover 32 and an air duct cover 33 adapted and connected to the front cover 32, an acceleration flow passage is formed between the front cover 32 and the air duct cover 33, an outlet of the blower fan 31 communicates with the acceleration flow passage, and an air outlet 34 of the acceleration flow passage is configured into a slit shape, and an air flow blown by the blower fan 31 flows through the acceleration flow passage and is accelerated to flow out of the air outlet 34, so that a pressure difference is formed inside and outside the air vent 132, thereby introducing air from a periphery of the air vent 132 into the massage area to form air flow circulation.

It needs to be mentioned that in this embodiment, an air flow is generated in the acceleration flow passage by the blower fan 31, the air flow is accelerated via the slit-like air outlet 34 of the acceleration flow passage, if a flow rate is high, a pressure will be low according to the Bernoulli's principle, a low pressure is formed inside the air vent 132 by means of a high-speed air flow, so that air at a peripheral side of the air vent 132 is introduced into the massage area. Air is introduced into the massage area from the front end of the air vent 132 and flows out through the hand inlet 131, thus forming continuous air flow circulation. Thus, a cool air flow is generated in the massage area, and the problems such as stuffiness and burning hot during massage are effectively relieved by blowing air to cool the temperature. Especially when a weather is hot, for people with more sweaty hands, the air flow carries away heat from the hands, making the hands feel cool and comfortable. In addition, the air flow circulation enables ventilation in the massage area, which can take away heat generated by the operation of the massager and avoid a stuffy feeling caused by the accumulation of hot air. A user can enjoy the massage while feeling the cool air flow, making the entire use process more comfortable.

In a preferred embodiment, a middle part of the front cover 32 and a middle part of the air duct cover 33 are provided with an air inlet 35 communicating with the air vent 132, the acceleration flow passage is annularly disposed at a front end of the air vent 132, and the air outlet 34 is disposed toward an inner side of the air vent 132. The air duct cover 33 is adapted and connected to the support 13, and one end of the air duct cover 33 extends into the air vent 132, and the other end of the air duct cover 33 protrudes around the air inlet 35 to form a first air outlet portion 331, and the first air outlet portion 331 is at least partially arranged to be bent from a side close to the air inlet 35 to a side away from the air inlet 35. The front cover 32 is provided with a second air outlet portion 321 protruding around a periphery of the air inlet 35, the second air outlet portion 321 is configured to be adapted to the first air outlet portion 331 to form the air outlet 34, and a plurality of fins 322 configured to abut against a curved portion of the first air outlet portion 331 are arranged at an inner side of the second air outlet portion 321 at intervals such that the fins 322 divide the air outlet 34 into a plurality of gas passages, further accelerating the flow of an air flow. Preferably, the front cover 32 is concavely provided with an arc-shaped groove adapted to the air duct cover 33 to form the acceleration flow passage, the air outlet 34 has a width of 1-2 mm, the air outlet 34 is disposed around the air vent 132, and the fins 322 are equidistantly distributed along the air outlet 34 to make air outlet uniform and ensure that the introduced air flows uniformly on a hand palm side and a hand back side. In another embodiment, the air outlet 34 is disposed in the form of a drum on the surface of an aircraft wing to accelerate an air flow, forming a pressure difference. A decorative cover 14, the front cover 32, the air duct cover 33, and the support 13 are connected in sequence by fasteners, which is not limited thereto and is not specifically limited.

On the basis of the above embodiment, in an alternative embodiment of the present disclosure, the massage mechanism includes an upper air bag 21, a lower air bag 22 and a massage head 23 which are disposed inside the support 13, the upper air bag 21 and the lower air bag 22 are connected to an air pump 25 via an air tube 24, and the massage head 23 is adapted and connected to a motor 26 via a connecting piece which may be a gear set or an assembly combined with a cam. The support 13 is configured to be wrapped with a cloth cover and place a hand of a user. The blower fan 31, the air pump 25 and the motor 26 are electrically connected to a control 41, and the control 41 is operatively triggered to drive the blower fan to blow air, the upper air bag and the lower air bag 22 to inflate and deflate, and the massage head 23 to rotate or swing up and down. The massage head 23 is arranged on the same side as the lower air bag 22, the massage head 23 is provided with a plurality of massage parts 231 in an array in a circumferential direction, and the lower air bag 22 is provided with a plurality of convex parts 221 facing an inner side of the massage area. Preferably, the massage head 23 is configured corresponding to the position of a palm of a hand and is configured to knead the palm of the hand to achieve the hand palm massage function. A wrist air bag 27 is disposed at the hand inlet 131, and the wrist air bag 27 is connected to the air pump 25, and inflation and deflation of the wrist air bag 27 is controlled by the air pump 25 for limiting a hand while massaging a wrist.

It should be mentioned that the control 41 is also electrically connected to a battery 43 and triggered via a button 42 to control the operation of the massage mechanism and the blowing mechanism, wherein the control 41 may be a circuit board, and a circuit program and a circuit principle of the circuit board are known in the art and will not be described in detail herein. A user performs opening or closing or selects the massage time and intensity of the massage mechanism, and an air speed of the blowing mechanism, etc. by pressing the button 42. The control 41 may also be triggered by a control panel in other embodiments, which is not specifically limited.

The above are only preferred embodiments of the present disclosure, and the scope of protection of the present disclosure is not limited to the above embodiments, and all technical solutions under the idea of the present disclosure fall within the scope of protection of the present disclosure.

What is claimed is:

1. A hand massager with a blowing function, comprising:
a body, comprising an upper housing, a lower housing, and a support disposed between the two housings, wherein both ends of the support is provided with an inlet for allowing a hand to enter and an air vent located at a finger end, and a massage area is formed between the inlet and the air vent;
a massage mechanism disposed in the massage area and configured to massage the hand; and
a blowing mechanism, mounted close to a position of the air vent, and comprising a blower fan, a front cover and an air duct cover adapted and connected to the front cover, wherein an acceleration flow passage is formed between the front cover and the air duct cover, an outlet of the blower fan communicates with the acceleration flow passage, an air outlet of the acceleration flow passage is constructed into a slit shape, and an air flow blown by the blower fan flows through the acceleration flow passage and is accelerated to flow out of the air outlet, so that a pressure difference is formed inside and outside the air vent, thereby introducing air from a periphery of the air vent into the massage area to form air flow circulation.

2. The hand massager with the blowing function according to claim 1, wherein a middle part of the front cover and a middle part of the air duct cover are provided with an air inlet communicating with the air vent in a penetrating manner, the acceleration flow passage is annularly disposed at a front end of the air vent, and the air outlet is disposed toward an inner side of the air vent.

3. The hand massager with the blowing function according to claim 2, wherein the air duct cover is adapted and connected to the support, and one end of the air duct cover extends into the air vent, and the other end of the air duct cover protrudes around the air inlet to form a first air outlet portion, and the first air outlet portion is at least partially arranged to be bent from a side close to the air inlet to a side away from the air inlet.

4. The hand massager with the blowing function according to claim 3, wherein the front cover is provided with a second air outlet portion protruding around a periphery of the air inlet, the second air outlet portion is configured to be adapted to the first air outlet portion to form the air outlet, and a plurality of fins configured to abut against a curved portion of the first air outlet portion are arranged at an inner side of the second air outlet portion at intervals such that the fins divide the air outlet into a plurality of gas passages.

5. The hand massager with the blowing function according to claim 4, wherein the air outlet has a width of 1-2 mm.

6. The hand massager with the blowing function according to claim 1, wherein the massage mechanism comprises an upper air bag, a lower air bag and a massage head which are disposed inside the support, the upper air bag and the lower air bag are connected with an air pump via an air tube, and the massage head is adapted and connected to a motor via a connecting piece.

7. The hand massager with the blowing function according to claim 6, wherein the blower fan, the air pump and the motor are electrically connected to a control, and the control is operatively triggered to drive the blower fan to blow air, the upper air bag and the lower air bag to inflate and deflate, and the massage head to rotate or swing up and down.

8. The hand massager with the blowing function according to claim 6, wherein the massage head is arranged on the same side as the lower air bag, the massage head is provided with a plurality of massage parts in an array in a circumferential direction, and the lower air bag is provided with a plurality of convex parts facing an inner side of the massage area.
